(19) United States
(12) Patent Application Publication
(10) Pub. No.: US 2014/0052216 A1
(43) Pub. Date: Feb. 20, 2014

(54) METHODS FOR PROMOTING WOUND HEALING

(71) Applicants: Gary L. Long, Cincinnati, OH (US); Gregory J. Bakos, Mason, OH (US); David N. Plescia, Mentor, OH (US); Peter K. Shires, Hamilton, OH (US)

(72) Inventors: Gary L. Long, Cincinnati, OH (US); Gregory J. Bakos, Mason, OH (US); David N. Plescia, Mentor, OH (US); Peter K. Shires, Hamilton, OH (US)

(73) Assignee: Ethicon Endo-Surgery, Inc., Cincinnati, OH (US)

(21) Appl. No.: 13/586,439

(22) Filed: Aug. 15, 2012

(51) Int. Cl. A61N 1/00

(52) U.S. Cl. 607/50

(57) ABSTRACT

A method for promoting wound healing at a wound site includes subjecting the wound site to electrical pulses to promote wound healing during at least one of the stages of wound healing. The method may further include closing the wound site by sutures or staples prior to and or after applying the electrical pulses that promote wound healing.

### BACKGROUND

As is well known, the healing of wounds in tissue such as skin generally involves, at least in adult humans and other mammals, a process of extra-cellular matrix (ESC) biosynthesis, turnover and organization which commonly leads to the production of fibrous, connective tissue scars and consequential loss of normal tissue function.

In the realm of surgery scar tissue formation and contraction is a major clinical problem for which there is no entirely satisfactory solution at present. Likewise, scarring and fibrosis following accidental burning or other injuries or trauma, particularly in children, often has serious results, leading to impaired function, defective future growth, and to unsightly aesthetic effects, and again presents a major problem.

In regard to unsightly aesthetic effects produced by scars, there also commonly arises a need for cosmetic treatment or operations to attempt to remove these disfigurements in order to improve appearance. Additionally, a similar need for cosmetic treatment often arises in connection with unwanted tattoos and other skin blemishes. At present, however, it is difficult or impossible to carry out such cosmetic treatment or operations satisfactorily since a certain amount of surgery is generally involved which in itself is likely to result in wounds producing fresh unsightly scar tissue.

Additionally, internal wounds generally caused during a surgical procedure, for example, to gain access to a surgical site, are stapled or sutured and left to heal over long periods of time, sometimes leaving the patient hospitalized longer than necessary, and possibly in pain for prolonged periods even after leaving the hospital. There is a clear need for improving the natural process of wound healing regardless of the wound site.

### FIGURES

The novel features of the various embodiments of the invention are set forth with particularity in the appended claims. The various embodiments of the invention, however, both as to organization and methods of operation, together with further objects and advantages thereof, may best be understood by reference to the following description, taken in conjunction with the accompanying drawings as follows.

FIG. 1 illustrates the stages of wound healing.

FIG. 2 illustrates an electrosurgical device according to certain embodiments described herein.

FIG. 3 illustrates an electrosurgical device according to certain embodiments described herein.

FIG. 4 illustrates an electrosurgical device including sensors according to certain embodiments described herein.

FIG. 5 illustrates an electrosurgical device including a temperature sensor according to certain embodiments described herein.

FIG. 6A is a graphical representation of monopolar electrical pulses that may be applied to a wound site according to certain embodiments described herein.

FIG. 6B is a graphical representation of bipolar electrical pulses that may be applied to a wound site according to certain embodiments described herein.

FIG. 7 is a graphical representation of electrical pulses that may be applied to a wound site according to certain embodiments described herein.

FIG. 8 is a graphical representation of electrical pulses that may be applied to a wound site according to certain embodiments described herein.

FIG. 9 is a graphical representation of electrical pulses that may be applied to a wound site according to certain embodiments described herein.

FIG. 10 is a graphical representation of an AC waveform that may be applied to a wound site according to certain embodiments described herein.

FIG. 11 is a graphical representation of a series of electrical pulses that may be applied to a wound site according to certain embodiments described herein.

FIG. 12 is a graphical representation of multiple bursts that may be applied to a wound site according to certain embodiments described herein.

FIG. 13 is a graphical representation of a treatment regimen generated and delivered to a wound site according to certain embodiments described herein.

FIG. 14 is a graphical representation of a treatment regimen generated and delivered to a wound site according to certain embodiments described herein.

FIG. 15 is a graphical representation of a treatment regimen generated and delivered to a wound site according to certain embodiments described herein.

FIG. 16 is an illustration of a method for promoting wound healing at a wound site in accordance with certain embodiments described herein.

FIG. 17 is an illustration of a method for promoting wound healing at a wound site in accordance with certain embodiments described herein.

FIG. 18 is an illustration of a method and a system for promoting wound healing at a wound site in accordance with certain embodiments described herein.

FIG. 19 is an illustration of a method and a system for promoting wound healing at a wound site in accordance with certain embodiments described herein

### SUMMARY

An aspect of the present disclosure is directed to a method for promoting wound healing in a patient. The method includes positioning first and second electrodes at or near a wound site. The method further comprises applying electrical pulses to tissue at the wound site, wherein the electrical pulses induce Irreversible Electroporation in cell membranes of the tissue at the wound site. Another aspect of the present disclosure is directed to a method for promoting wound healing in a patient, the method comprising subjecting a wound site to electrical pulses that promote formation of a Hemostatic plug at the wound site. Yet another aspect of the present disclosure is directed to a method for promoting wound healing in a patient, the method comprising applying electrical pulses to a wound site, wherein the electrical pulses sterilize the wound site by inducing Irreversible Electroporation in foreign microorganisms at the wound site.

An aspect of the present disclosure is directed to a method for treating a wound site in a patient. The method includes operating a surgical stapler to close the wound site by deploying staples across the wound site, and applying electrical pulses to the wound site through the staples.

An aspect of the present disclosure is directed to an electrosurgical system for treating a wound site in a patient. The electrosurgical system includes an energy source, and at least one staple deployable at the wound site, wherein the at least one staple is electrically coupled to the energy source, and wherein the at least one staple is configured to deliver energy from the energy source to tissue in electrical contact therewith.

### DESCRIPTION

Applicant of the present application also owns U.S. patent application Ser. No. ______, entitled “ELECTROSURGICAL DEVICES AND METHODS,” (Attorney Docket No. END7118USNP/120084), which has been filed on even date herewith, and which is herein incorporated by reference in its entirety.

The present disclosure directed to electrosurgical apparatuses, systems, and methods for promoting wound healing and facilitating repair and healing of animal tissue, especially, but not exclusively, skin or other epithelial tissue, that has been damaged by, for example, wounds resulting from accidental injury, burn, surgical operations, or other trauma.

The present disclosure describes various elements, features, aspects, and advantages of various embodiments of electrosurgical devices and methods thereof. It is to be understood that certain descriptions of the various embodiments have been simplified to illustrate only those elements, features and aspects that are relevant to a more clear understanding of the disclosed embodiments, while eliminating, for purposes of brevity or clarity, other elements, features and aspects. Any references to “various embodiments,” “some embodiments,” “one embodiment,” or “an embodiment” generally means that a particular element, feature and/or aspect described in the embodiment is included in at least one embodiment. The phrases “in various embodiments,” “in some embodiments,” “in one embodiment,” or “in an embodiment” may not refer to the same embodiment. Persons having ordinary skill in the art, upon considering the description herein, will recognize that various combinations or sub-combinations of the various embodiments and other elements, features, and aspects may be desirable in particular implementations or applications. However, because such other elements, features, and aspects may be readily ascertained by persons having ordinary skill in the art upon considering the description herein, and are not necessary for a complete understanding of the disclosed embodiments, a description of such elements, features, and aspects may not be provided. As such, it is to be understood that the description set forth herein is merely an illustrative example of the disclosed embodiments and is not intended to limit the scope of the invention as defined solely by the claims.

All numerical quantities stated herein are approximate unless stated otherwise, meaning that the term “about” may be inferred when not expressly stated. The numerical quantities disclosed herein are to be understood as not being strictly limited to the exact numerical values recited. Instead, unless stated otherwise, each numerical value is intended to mean both the recited value and a functionally equivalent range surrounding that value. At the very least, and not as an attempt to limit the application of the doctrine of equivalents to the scope of the claims, each numerical parameter should at least be construed in light of the number of reported significant digits and by applying ordinary rounding techniques. Notwithstanding the approximations of numerical quantities stated herein, the numerical quantities described in specific examples of actual measured values are reported as precisely as possible.

All numerical ranges stated herein include all sub-ranges subsumed therein. For example, a range of “1 to 10” is intended to include all sub-ranges between and including the recited minimum value of 1 and the recited maximum value of 10. Any maximum numerical limitation recited herein is intended to include all lower numerical limitations. Any minimum numerical limitation recited herein is intended to include all higher numerical limitations.

As generally used herein, the terms “proximal” and “distal” generally refer to a clinician manipulating one end of an instrument used to treat a patient. The term “proximal” generally refers to the portion of the instrument closest to the clinician. The term “distal” generally refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

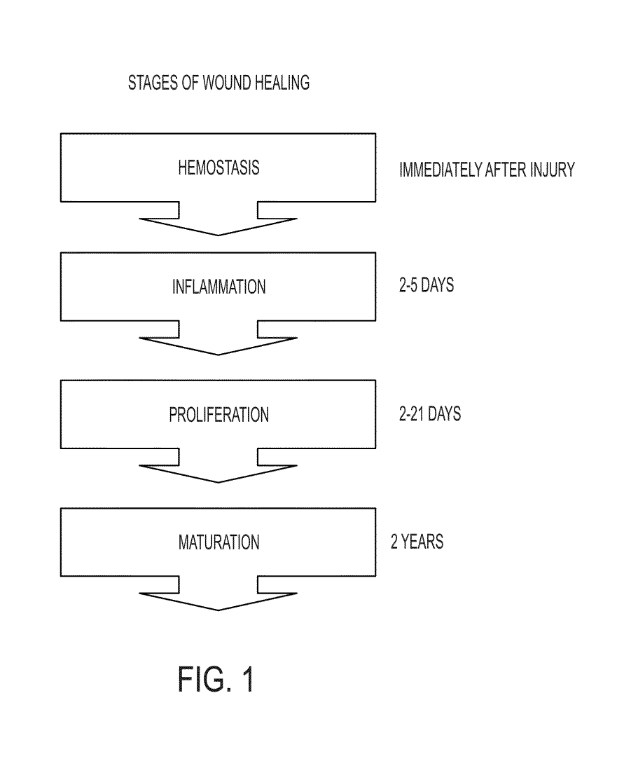

As illustrated in FIG. 1, the natural process of wound healing generally includes four main stages. The first stage is Hemostasis. Injured blood vessels in a wound site must be sealed. Blood vessels constrict in response to injury but this spasm ultimately relaxes. Blood platelets secrete vasoconstrictive substances to aid in this process but their prime role is to form a stable clot sealing the damaged vessels. Under the influence of adenosine diphosphate (ADP) leaking from damaged tissue, in the wound site, blood platelets aggregate, and adhere to exposed collagen. Blood platelets also secrete factors, which interact with and stimulate and intrinsic clotting cascade through the production of thrombin, which in turn initiates the formation of fibrin from fibrinogen. Fibrin mesh strengthens the platelet aggregate into a stable hemostatic plug. Additionally, platelets also secrete cytokines such as platelet-derived growth factor (PDGF), which is recognized as one of the first factors secreted in initiating subsequent stages. Hemostasis generally occurs within minutes of the initial injury unless there are underlying clotting disorders.

The second stage of the natural process of wound healing is an inflammatory stage. This stage usually lasts for up to 2-5 days post injury. The inflammatory stage is the body's natural response to injury. Blood vessel walls dilate to allow essential cells, antibodies, growth factors, enzymes, and nutrients to reach the wounded area. This leads to a rise in exudate levels. It is at this stage that characteristic signs of inflammation can be seen; erythema, heat, oedema, pain, and functional disturbance often last for up to 4 days post injury. The predominant cells at work here are the phagocytic cells, neutrophils and macrophages. Neutrophils phagocytize debris and microorganisms, and provide a first line of defense against infection. They are aided by local mast cells. Fibrin is broken down, and degradation products attract Macrophages, which are able to phagocytize bacteria and provide a second line of defense. Macrophages also secrete a variety of chemotactic factors and growth factors such as fibroblast growth factor (FGF), epidermal growth factor (EGF), transforming growth factor beta (TGF-β), and interleukin-1 (IL-1) which appears to direct remaining stages.

The remaining stages in the natural process of wound healing are proliferation followed by maturation. The proliferation stage usually lasts for up to 2-21 days and includes granulation and wound contraction; the maturation phase may last for two years following an injury. During the proliferation stage, the wound is ‘rebuilt’ with new granulation tissue which is comprised of collagen and extracellular matrix into which a new network of blood vessels develop, a process known as ‘angiogenesis’. keratinocytes then resurface the wound, a process known as ‘epithelialization”. In the final stage of epithelialization, contracture occurs as the keratinocytes differentiate to form the protective outer layer or stratum corneum. Contraction is a key phase of wound healing. In full thickness wounds, contraction peaks at about 5 to 15 days post wounding. Contraction can last for several weeks and continues even after the wound is completely reepithelialized. Contraction is the main cause of scarring associated with wound healing. Maturation is the final phase and occurs once the wound has closed. Maturation involves remodeling dermal tissues to produce greater tensile strength. The principle cell involved in this process is fibroblast. Cellular activity is reduced and number of blood vessels in a wounded area regress and decrease. Remodeling can take up to 2 years.

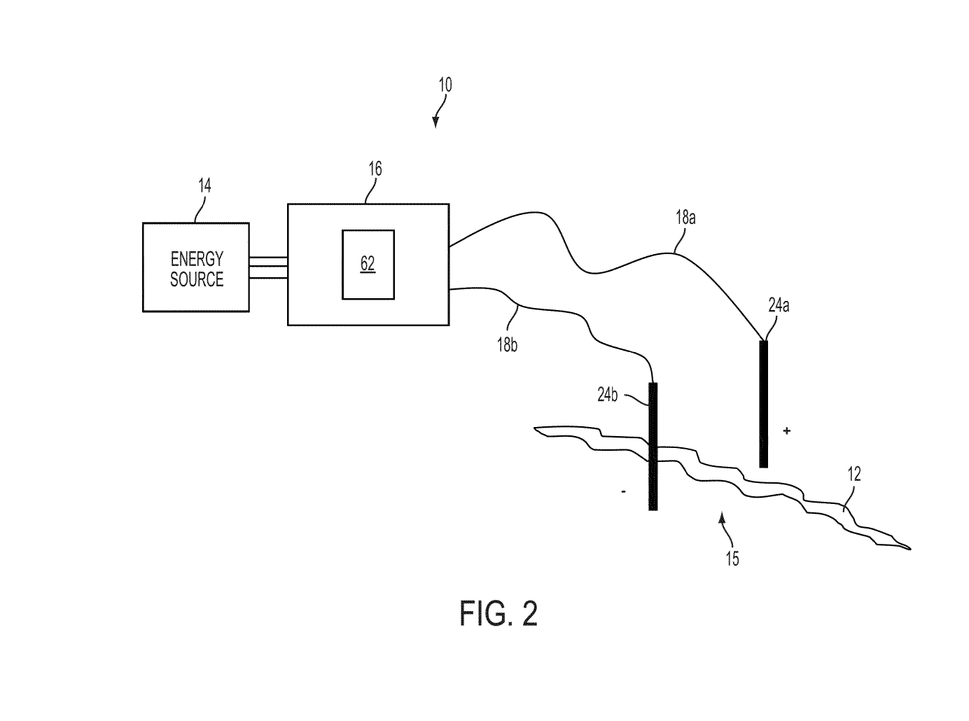

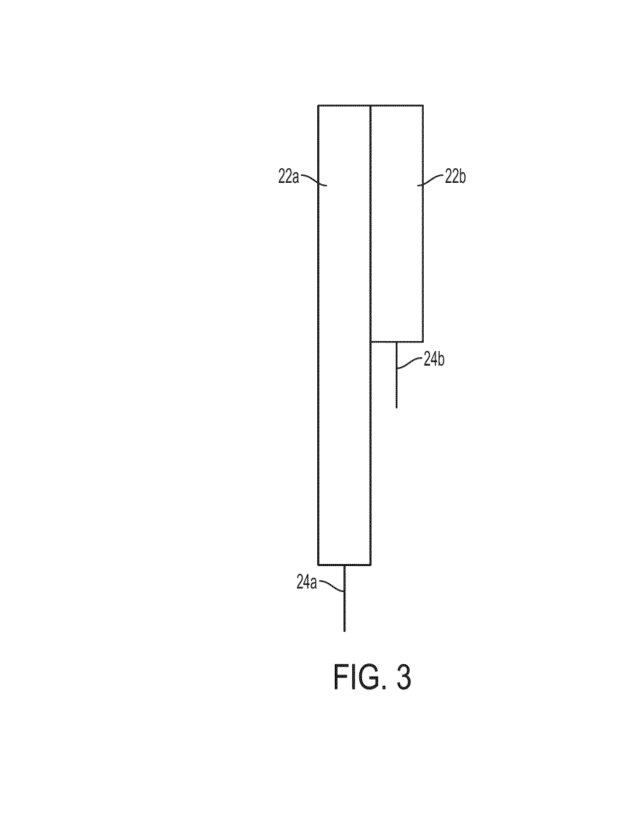

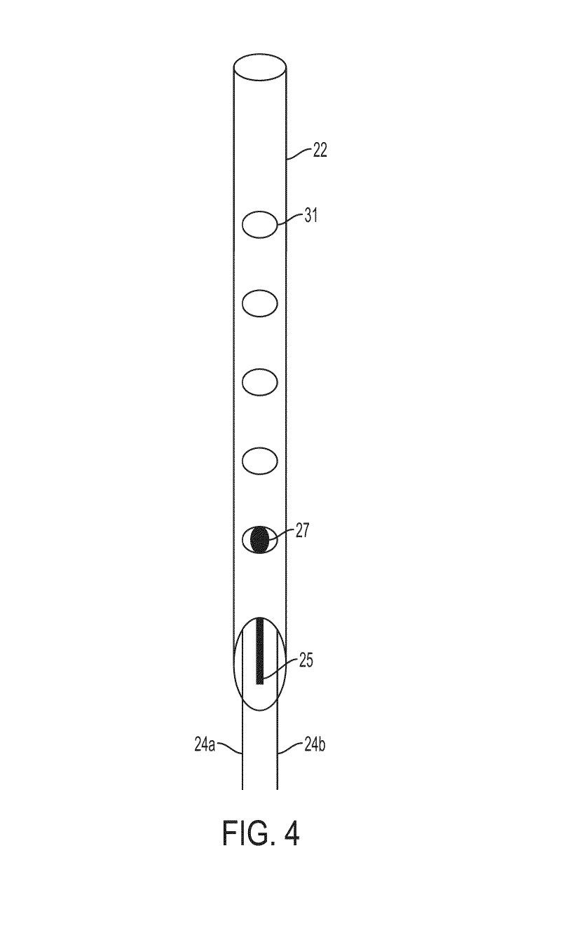

Referring to FIG. 2-5, an electrosurgical system 10 is used in a method for promoting wound healing at a wound site 12, for example, on a skin surface 15. The electrosurgical system 10 may comprise an energy source 14 coupled to a first electrode 24a and coupled to a second electrode 24b. The method for promoting wound healing may comprise positioning the first electrode 24a, and the second electrode 24b at or near the wound site 12 as illustrated in FIG. 2. The method may comprise operating the electrosurgical system 10 to apply electrical pulses 70 to tissue at the wound site 12. The electrical pulses 70 may promote wound healing at wound site 12.

Without wising to be bound to a particular theory, the electrical pulses 70 may promote wound healing by promoting Hemostasis. As described above, blood platelets play a significant role in Hemostasis following an injury. The electrical pulses 70 may promote wound healing at wound site 12 by temporarily increasing the permeability of blood vessel walls in and around the wound site 12. This, in turn, may temporarily increase the escape of blood cells including platelets. Increased platelets count at the wound site 12 may expedite formation of a hemostatic plug. In certain embodiments, the electrical pulses 70 may promote the formation of a hemostatic plug while causing no or minimal thermal damage to extracellular matrix and blood vessels at the wound site 12. In certain embodiments, the electrical pulses 70 may promote the formation of a hemostatic plug while maintaining tissue temperature at the wound site 12 below a maximum temperature. The maximum temperature may be equal to, or less than 60° C.

In at least one embodiment, the electrical pulses 70 may promote wound healing at wound site 12 by sterilizing wound site 12 thereby killing foreign microorganisms at the wound site 12, which reduces the risk of infection. For example, the electrical pulses 70 may kill bacteria at the wound site 12. The electrical pulses 70 may kill foreign microorganisms such as bacteria by inducing Irreversible Electroporation in the membranes of bacteria. The electrical pulses 70 may sterilize wound site 12 while causing no or minimal thermal damage to extracellular matrix and blood vessels at the wound site 12. In at least one embodiment, the electrical pulses sterilize wound site 12 while maintaining native tissue temperature in and around the wound site 12 below a maximum temperature. In at least one embodiment, the maximum temperature may be equal to, or less than 60° C.

In at least one embodiment, the electrical pulses 70 may promote wound healing at wound site 12 by increasing Neutrophils count at the wound site 12. The electrical pulses 70 may temporarily increase the permeability of blood vessel walls at wound site 12, as described above, thereby increasing the escape of Neutrophils. An increased Neutrophils count at the wound site 12 may improve the natural process of wound healing, for example, by expediting phagocytosis of debris and microorganisms.

In at least one embodiment, the electrical pulses 70 may promote wound healing at wound site 12 inducing Irreversible Electroporation in the membranes of native tissue cells at the wound site 12, thereby releasing large amounts of chemotactic agents into the wound site 12, which may expedite the attraction of appropriate responding cells such as but not limited to a number of inflammatory cells. In result, the usual slow erythema and swelling associated with the inflammatory stage may be preempted or reduced, and in absence of infection causing microorganisms, wound healing may be expedited.

In at least one embodiment, the electrical pulses 70 may promote wound healing at wound site 12 by causing no or minimal thermal damage to extra-cellular matrix and blood vessels at the wound site 12. Sterilizing the wound site and/or enhancing signaling pathways while leaving collagen framework and blood vessels mainly intact may signal local environment at the wound site 12 to pursue a process of regeneration instead of wound healing. Regeneration, unlike typical wound healing, does not require fibroblast proliferation, or excessive collagen deposition, which generally cause tissue contraction and scarring during typical wound healing. Furthermore, with extra-cellular matrix and blood vessels mostly intact, the sacrificing of undamaged healthy tissue to produce a wound healing environment may be eliminated or reduced. In result, pulsed tissue can almost promptly begin to repopulate with healthy, vascularized, regenerative tissue in the same design as originally present.

In various embodiments, the electrosurgical system 10 may be configured to generate and deliver electrical pulses 70 that induce Irreversible Electroporation at wound site 12 as described above. The electrosurgical system 10 may be configured to induce Irreversible Electroporation at wound site 12 in a controlled and focused manner without inducing thermally damaging effects to the surrounding tissue. Electroporation, or electropermeabilization, is a significant increase in the electrical conductivity and permeability of a cell plasma membrane caused by an externally applied electrical field. The external electric field (electric potential per unit length) to which the cell membrane is exposed significantly increases the electrical conductivity and permeability of the plasma in the cell membrane. The primary parameter affecting the transmembrane potential is the potential difference across the cell membrane. Irreversible electroporation is the application of an electric field of a specific magnitude and duration to a cell membrane such that the permeabilization of the cell membrane cannot be reversed, leading to cell death without inducing a significant amount of heat in the cell membrane. The destabilizing potential forms pores in the cell membrane when the potential across the cell membrane exceeds a threshold causing the cell to die.

Without wishing to be bound to any particular theory, cell death due to Irreversible Electroporation may occur directly following the treatment. Alternatively, cell death may occur later due to various biological mechanisms. In one theory, Irreversible Electroporation may cause cell death under a process known as necrosis. It is believed that each cell type has a necrotic threshold. The necrotic threshold generally refers the electric field strength that induces cell necrosis by Irreversible Electroporation. The necrotic threshold may relate to at least the following parameters: cell type, temperature, electrical conductivity, pH and tissue perfusion. In another theory, cell death may occur due a process known as apoptosis. Apoptosis is programmed cell death. Apoptosis involves a series of biochemical events that lead to a variety of morphological changes, including changes to the cell membrane such as loss of membrane asymmetry and attachment, cell shrinkage, nuclear fragmentation, chromatin condensation, and chromosomal Deoxyribonucleic acid (DNA) fragmentation.

As described above, the application of the electric pulses 70 to cells at a wound site can be an effective way for causing the local tissue cells to die without deleterious thermal effects to the surrounding healthy tissue associated with thermal-inducing ablation treatments. The electric pulses 70 may destroy cells without heat and thus do not destroy the cellular support structure or regional vasculature.

Referring to FIG. 2-5, once the first electrode 24a, and the second electrode 24b are positioned into or proximal to wound site 12, an energizing potential may be applied to the electrodes to create an electric field to which the wound site 12 is exposed. The energizing potential (and the resulting electric field) may be characterized by multiple parameters such as frequency, amplitude, pulse width (duration of a pulse or pulse length), and/or polarity. Depending on the treatment to be rendered, a particular electrode may be configured either as an anode (+) or a cathode (−) or may comprise a plurality of electrodes with at least one configured as an anode and at least one other configured as a cathode. Regardless of the initial polar configuration, the polarity of the electrodes may be reversed by reversing the polarity of the output of the energy source 14.

In general, the first electrode 24a, and the second electrode 24b each comprise an electrically conductive portion (e.g., medical grade stainless steel) and are configured to electrically couple to energy source 14. Various electrode designs, suitable for use with the present disclosure, described in commonly-owned U.S. Patent Application Publication No. 2009/0182332 A1 titled “IN-LINE ELECTROSURGICAL FORCEPS,” filed Jan. 15, 2008, the entire disclosure of which is incorporated herein by reference in its entirety, and commonly-owned U.S. Patent Application Publication No. 2009/0112063 A1 titled “ENDOSCOPIC OVERTUBES,” filed Oct. 31, 2007, the entire disclosure of which is incorporated herein by reference in its entirety.

In various embodiments, as illustrated in FIG. 2, one or more electrodes (e.g., needle electrodes, balloon electrodes), such as first and second electrodes 24a,b may extend out from the distal end of the electrosurgical system 10. In one embodiment, the first electrode 24a may be configured as a positive electrode and the second electrode 24b may be configured as a negative electrode. The first electrode 24a may be electrically connected to a first electrical conductor 18a, or similar electrically conductive lead or wire, which may be coupled to a positive terminal of energy source 14 through an activation switch 62. The second electrode 24b may be electrically connected to a second electrical conductor 18b, or similar electrically conductive lead or wire, which may be coupled to a negative terminal of the energy source 14 through the activation switch 62. The electrical conductors 18a,b may be electrically insulated from each other and surrounding structures, except for the electrical connections to the respective electrodes 24a,b.

The electrodes 24a,b may have a diameter or radius from 0.5 mm to 1.5 mm, such as, for example, 0.5 mm, 0.75 mm, 1 mm, and 1.5 mm. In various embodiments, the diameter of the first electrode 24a may be different from the diameter of the second electrode 24b. The electrode spacing may be from 0.5 cm to 3 cm. In various embodiments, the distance from the first electrode 24a to the second electrode 24b may be from 0.5 cm to 3 cm, such as, for example, 1 cm, 1.5 cm, 2.0 cm, and 3 cm. In one embodiment, the electrosurgical system 10 may comprise multiple needle electrodes.

FIG. 2 illustrates the first 24a and second 24b electrodes in use to treat wound site 12. The first 24a and second 24b electrodes are embedded into or proximate the wound site 12 on skin surface 15. The first 24a and second 24b electrodes are energized to deliver electrical pulses 70 of amplitude and length sufficient to promote wound healing at wound site 12. Varying the size and spacing of the first 24a and second 24b electrodes can control the size and shape of the treatment zone. In addition, Electric pulse amplitude and length can be varied to control the size and shape of the treatment zone.

Various electrosurgical systems and instruments are disclosed in commonly-owned U.S. Patent Application Publication No. 2009/0062788 A1 titled “ELECTRICAL ABLATION SURGICAL INSTRUMENTS,” filed Aug. 31, 2007, the entire disclosure of which is incorporated herein by reference in its entirety. Various electrode designs are disclosed in commonly-owned U.S. Patent Application Publication No. 2010/0179530 A1, titled “ELECTRICAL ABLATION DEVICES”, filed on Jan. 12, 2009, the entire disclosure of which is incorporated herein by reference in its entirety.

In at least one embodiment, the energy source 14 may comprise a wireless transmitter to deliver energy to the electrodes 24a and 24b using wireless energy transfer techniques via one or more remotely positioned antennas. Those skilled in the art will appreciate that wireless energy transfer or wireless power transmission is a process of transmitting electrical energy from an energy source to an electrical load without interconnecting wires. An electrical transformer is the simplest instance of wireless energy transfer. The primary and secondary circuits of a transformer are not directly connected and the transfer of energy takes place by electromagnetic coupling through a process known as mutual induction. Power also may be transferred wirelessly using RF energy. Wireless power transfer technology using RF energy is produced by Powercast, Inc. and can achieve an output of 6 volts for a little over one meter. Other low-power wireless power technology has been proposed such as described in U.S. Pat. No. 6,967,462, the entire disclosure of which is incorporated herein by reference.

In various embodiments, energy source 14 may comprise an electrical waveform generator, which may be configured to generate electrical pulses 70, which are capable of promoting wound healing at wound site 12. The energy source 14 may be configured to generate electrical pulses 70 in the form of direct-current (DC) and/or alternating-current (AC) voltage potentials. The electrical pulses 70 may be characterized by various parameters such as frequency, amplitude, pulse length, and/or polarity.

In at least one embodiment, the first 24a and second 24b electrodes are adapted and configured to electrically couple to the energy source 14 (e.g., generator, waveform generator). Once electrical energy is transmitted to the first 24a and second 24b electrodes, an electric field is formed at a distal end of the first 24a and second 24b electrodes. The energy source 14 may be configured to generate electric pulses 70 at a predetermined frequency, amplitude, pulse length, and/or polarity that are suitable to promote wound healing at wound site 12. For example, the energy source 14 may be configured to deliver DC electric pulses 70 having a predetermined frequency, amplitude, pulse length, and/or polarity suitable to promote wound healing wound site 12. The DC pulses may be positive or negative relative to a particular reference polarity. The polarity of the DC pulses may be reversed or inverted from positive-to-negative or negative-to-positive a predetermined number of times to promote wound healing at wound site 12.

In at least one embodiment, a timing circuit may be coupled to the output of the energy source 14 to generate electric pulses 70. The timing circuit may comprise one or more suitable switching elements to produce the electric pulses 70. For example, the energy source 14 may produce a series of n electric pulses 70 (where n is any positive integer) of sufficient amplitude and duration to promote wound healing at wound healing 12. In at least one embodiment, the electric pulses 70 may have a fixed or variable pulse length, amplitude, and/or frequency.

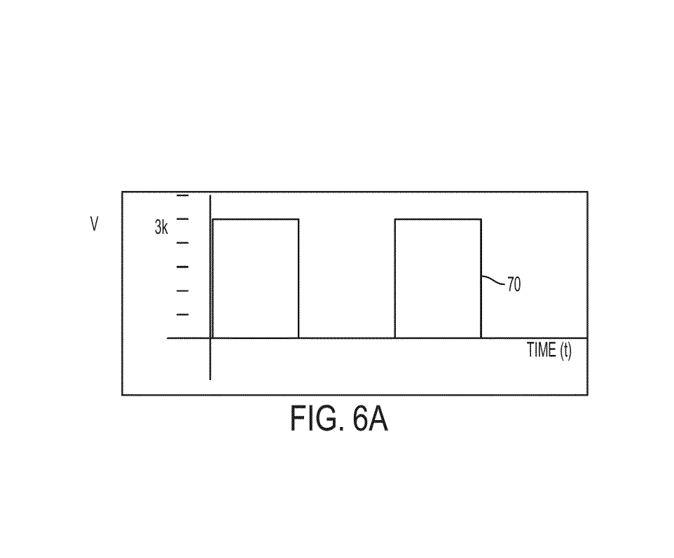

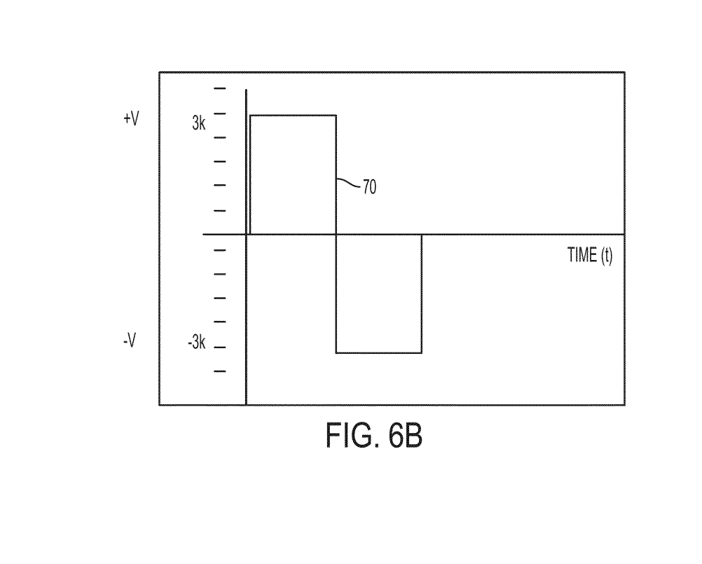

Referring to FIGS. 6A and 6B, the electrosurgical system 10 may promote wound healing at wound site 12 by generating and delivering electrical pulses 70 to the wound site 12 that are monopolar and/or bipolar pulses. FIG. 6A is a graphical representation of a series of monopolar electrical pulses 70, in accordance with certain embodiments described herein, having the same polarity in which each pulse has an amplitude of +3,000 VDC. In monopolar mode, a grounding pad may be substituted for one of the electrodes 24a, and 24b. FIG. 6B is a graphical representation of a series of bipolar electrical pulses 70, in accordance with certain embodiments described herein, having opposite polarity in which the first electrical pulse has an amplitude of +3,000 VDC and the second electrical pulse has an amplitude of −3,000 VDC. In bipolar mode, the polarity of the first 24a and second 24b electrodes may alternate. In bipolar mode, the first electrode 24a may be electrically connected to a first polarity and the second electrode 24b may be electrically connected to the opposite polarity. In monopolar mode, the first electrode 24a may be electrically connected to a prescribed voltage and the second electrode 24b may be set to ground. The energy source 14 may be configured to operate in either the bipolar or the monopolar modes. When more than two electrodes are used, the polarity of the electrodes may be alternated so that any two adjacent electrodes may have either the same or opposite polarities. In bipolar mode, the negative electrode of the energy source 14 may be coupled to an impedance simulation circuit.

In at least one embodiment, the energy source 14 may be configured to produce destabilizing electrical potentials (e.g., fields) suitable to induce Irreversible Electroporation. The destabilizing electrical potentials may be in the form of bipolar/monopolar DC electric pulses 70 suitable for promoting wound healing at wound site 12. A commercially available energy source suitable for generating Irreversible Electroporation electric filed pulses 70 in bipolar or monopolar mode is a pulsed DC generator such as Model Number ECM 830, available from BTX Molecular Delivery Systems Boston, Mass. In bipolar mode, the first electrode 24a may be electrically coupled to a first polarity and the second electrode 24b may be electrically coupled to a second (e.g., opposite) polarity of the energy source 14. Bipolar/monopolar DC electric pulses may be produced at a variety of frequencies, amplitudes, pulse lengths, and/or polarities.

In at least one embodiment, the energy source 14 can be configured to produce DC electric pulses 70 at frequencies in the range of approximately 1 Hz to approximately 10000 Hz, amplitudes in the range of approximately ±100 to approximately ±8000 VDC, and pulse width (duration) in the range of approximately 1 μs to approximately 100 ms. In at least one embodiment, the energy source 14 can be configured to produce biphasic waveforms and/or monophasic waveforms that alternate approximately 0V. In various embodiments, for example, the polarity of the electric potentials coupled to the electrodes 24a,b can be reversed during the treatment. For example, initially, the DC electric pulses 70 can have a positive polarity and an amplitude in the range of approximately +100 to approximately +3000 VDC. Subsequently, the polarity of the DC electric pulses 70 can be reversed such that the amplitude is in the range of approximately −100 to approximately −3000 VDC. In another embodiment, the DC electric pulses 70 can have an initial positive polarity and amplitude in the range of approximately +100 to +6000 VDC and a subsequently reversed polarity and amplitude in the range of approximately −100 to approximately −6000 VDC. The electrical pulses 70 may be delivered in bursts. The time between bursts may be in the range of about 0.001 seconds to about 100 seconds. The total number of pulses per burst may be in the range of about 1 to about 100. The total number of bursts may be in the range of about 1 burst to about 1000 bursts. It has been determined that an electric field strength of 800-1000V/cm can be suitable for destroying living tissue by inducing Irreversible Electroporation by DC electric pulses 70.

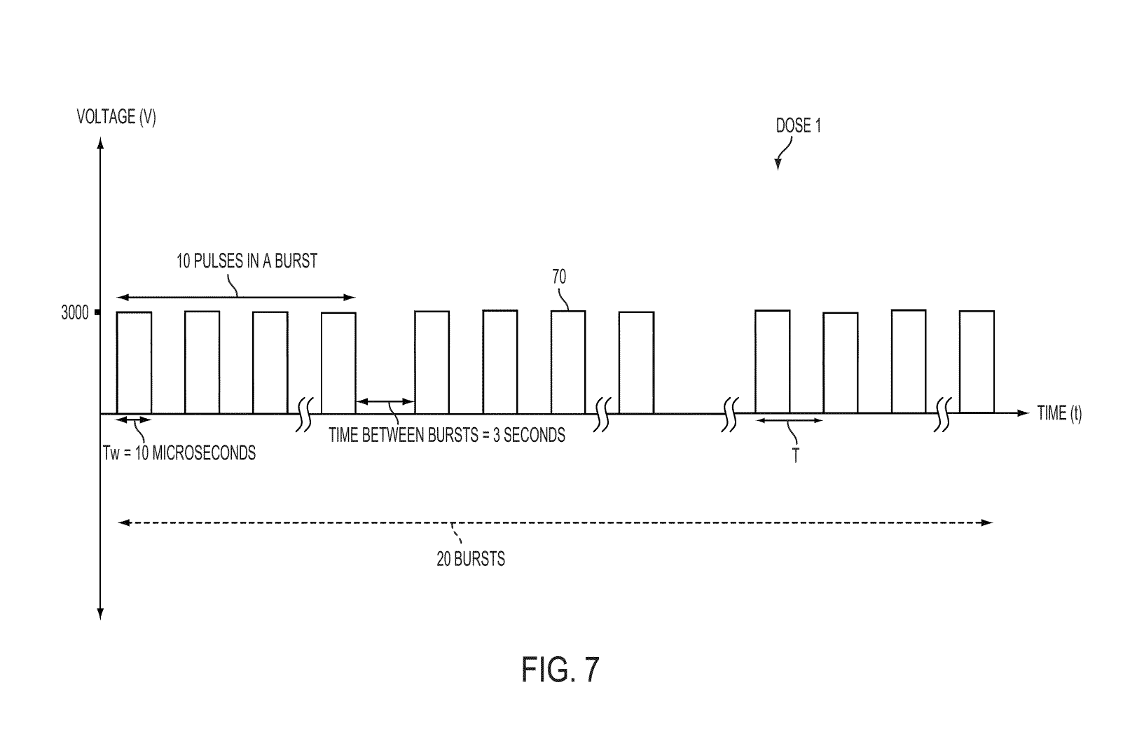

FIG. 7 is a graphical representation of a treatment regimen (Dose 1) used to promote wound healing at a wound site 12 in accordance with certain embodiments described herein. In this example, as illustrated in FIG. 7, Dose 1 includes several electrical pulses 70. Each pulse has an amplitude of approximately +3000 VDC and pulse duration T_{w} of approximately 10 μs delivered at a pulse period T or repetition rate, frequency f=1/T, of approximately 200 Hz. The electrical pulses 70 are delivered in bursts. Each burst includes 10 pulses. The time between bursts is 3 seconds. The total number of bursts in Dose 1 is 20. In this example, the electrodes 24a,b are spaced 1.5 cm apart.

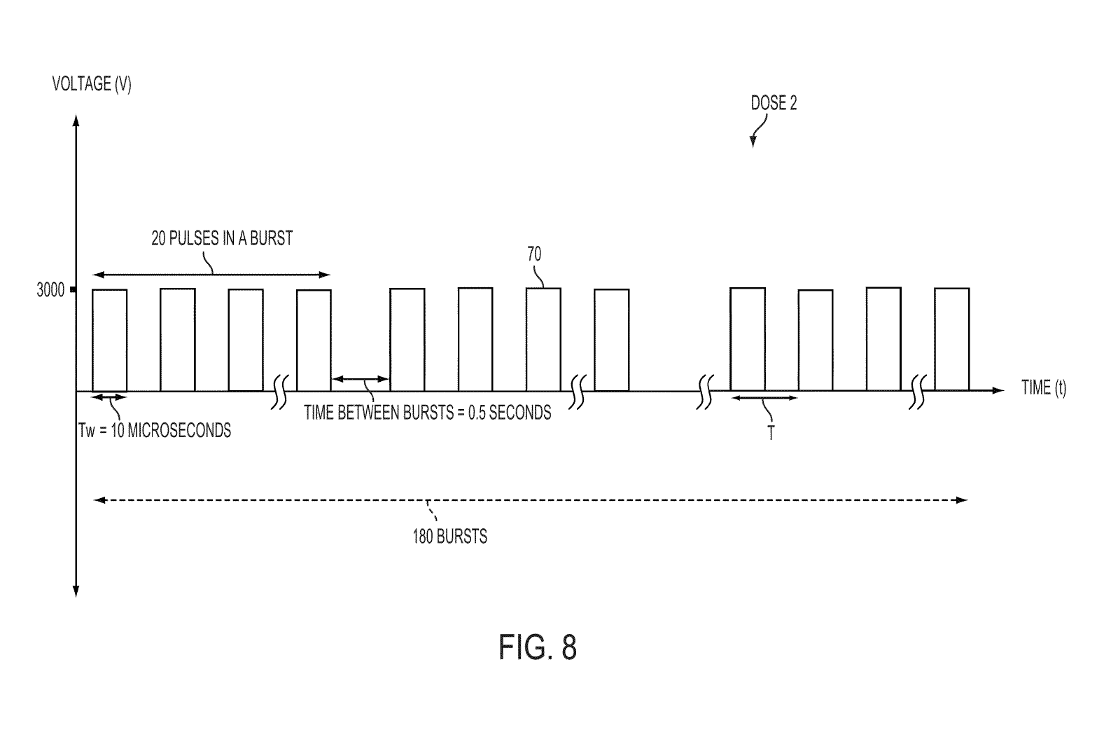

FIG. 8 is a graphical representation of a treatment regimen (Dose 2) used to promote wound healing at a wound site 12 in accordance with certain embodiments described herein. In this example, as illustrated in FIG. 8, Dose 2 includes several electrical pulses 70. Each pulse has an amplitude of approximately +3000 VDC and pulse duration T_{w} of approximately 10 μs delivered at a pulse period T or repetition rate, frequency f=1/T, of approximately 200 Hz. The electrical pulses 70 are delivered in bursts. Each burst includes 20 pulses. The time between bursts is 0.5 seconds. The total number of bursts in (Dose 2) is 180. In this example, the electrodes 24a,b are spaced 1.5 cm apart.

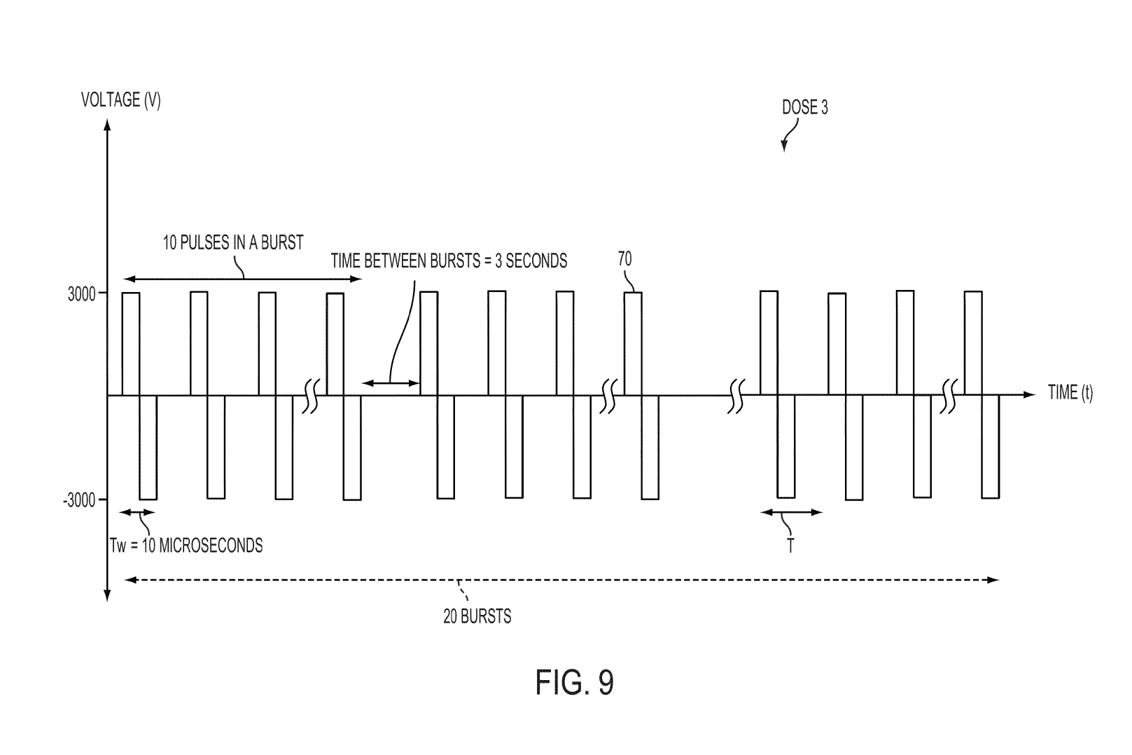

FIG. 9 is a graphical representation of a treatment regimen (Dose 3) used to promote wound healing at a wound site 12 in accordance with certain embodiments described herein. In this example, as illustrated in FIG. 9, Dose 3 includes several electrical pulses 70. Each pulse has a positive amplitude of approximately +3000 VDC, a negative amplitude of −3000 VDC, and pulse duration T, of approximately 10 μs (5 μs during the positive amplitude, and 5 μs during the negative amplitude) delivered at a pulse period T or repetition rate, frequency f=1/T, of approximately 200 Hz. The electrical pulses 70 are delivered in bursts. Each burst includes 10 pulses. The time between bursts is 3 seconds. The total number of bursts in (Dose 3) is 20. In this example, the electrodes 24a,b are spaced 1.5 cm apart.

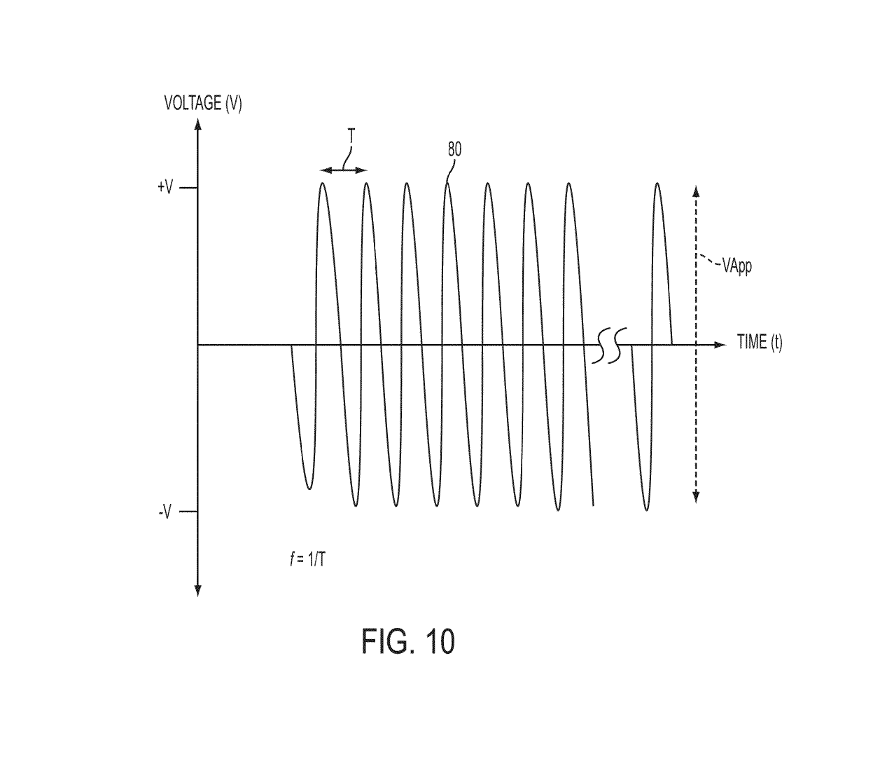

In various embodiments, energy source 14 may comprise an AC waveform generator. Energy source 14 may generate and deliver a radio frequency AC waveform 80, as illustrated in FIG. 10, to promote wound healing at a wound site 12. Time (t) is shown along the horizontal axis and voltage (VAC) is shown along the vertical axis. The AC waveform 80 has a fundamental frequency f, and a peak-to-peak voltage amplitude (VAₚₚ). In various embodiments, the AC waveform 80 may have a fundamental frequency f in the range of about 330 KHz to about 900 KHz, and peak-to-peak voltage amplitude (VAₚₚ) in the range of about 200 VAC to about 12,000 VAC. In other embodiments, the AC waveform 80 may have a fundamental frequency f in the range of about 400 KHz to about 500 KHz and peak-to-peak amplitude voltage (VAₚₚ) in the range of about 5,000 VAC to about 12,000 VAC. In one embodiment, the AC waveform 80 may have a fundamental frequency f of 500 KHz, and peak-to-peak voltage amplitude (VAₚₚ) of 12,000 VAC.

The energy source 14 may be configured to generate and deliver AC waveform 80 in pulses 70 to promote wound healing at a wound site 12 with no or minimal thermal damage to extracellular matrix and blood vessels. Each pulse may have a duration T_{w} delivered at a pulse period T₁ or a pulse frequency f1=1/T₁. A timing circuit may be coupled to the output of the energy source 14 to generate electric pulses. The timing circuit may comprise one or more suitable switching elements to produce the electric pulses.

The energy source 14 may be configured to generate and deliver AC waveform 80 in several bursts, each burst including several pulses 70. A treatment regimen may comprise several bursts spaced apart by sufficient time T_{b} to allow the temperature of the treated tissue to remain below a maximum temperature. The bursts may be delivered at a burst period T₂ or a burst frequency f₂=1/T₂. Both pulse and burst frequencies may be varied within a particular treatment regimen to effectively treat target tissue while maintaining treated tissue temperature below a maximum temperature.

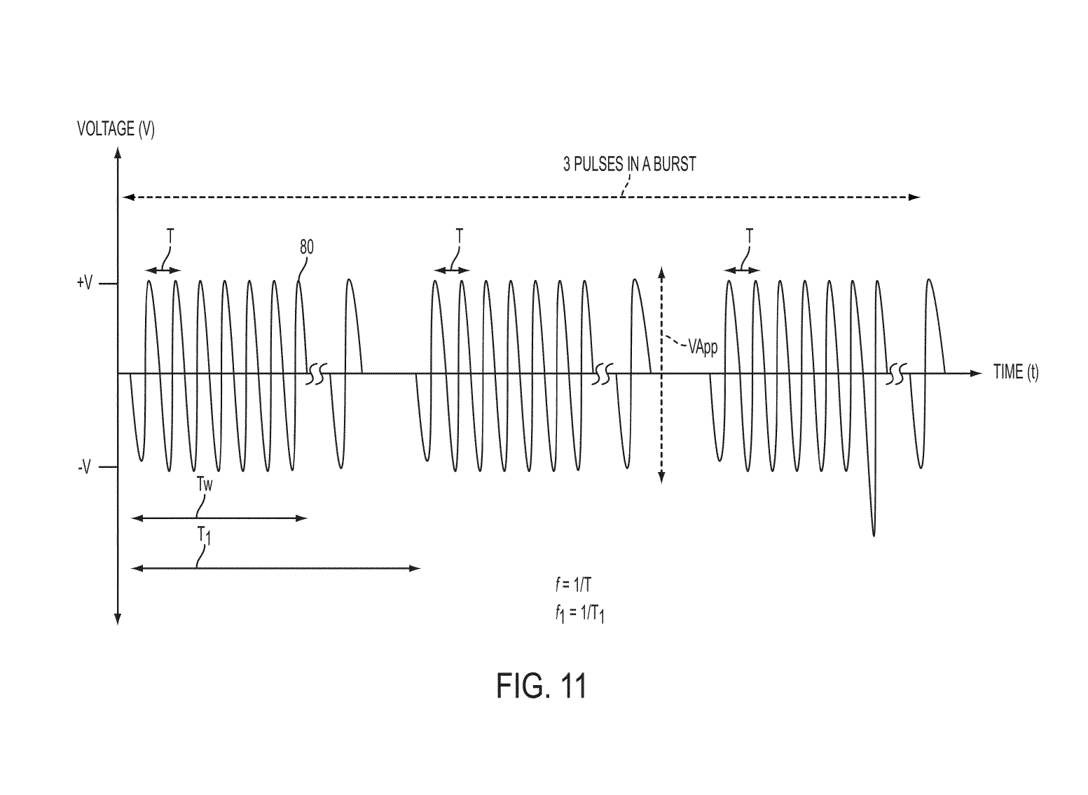

FIG. 11 is a graphical representation of a burst of electrical pulses 70 of AC waveform 80 generated and delivered to wound site 12 by energy source 14 to promote wound healing at wound site 12. Time (t) is shown along the horizontal axis and voltage (VAC) is shown along the vertical axis. Waveform 80 has a fundamental frequency f, and a voltage peak-to-peak amplitude (VAₚₚ). In this example, the burst includes three pulses 70. Each pulse has a duration T_{w} delivered at a pulse period T₁ or a pulse frequency f₁=1/T₁. One of ordinary skill in the art will appreciate that the total energy delivered by each burst to the tissue can be varied by changing the voltage peak-to-peak amplitude (VAₚₚ), and/or the fundamental frequency f, the pulse duration T_{w}, and/or the pulse frequency f₁.

In various embodiments, each pulse may have pulse duration T_{w} in the range of about 5 microseconds to about 100 microseconds. In other embodiments, each pulse 70 may have pulse duration T_{w} in the range of about 10 microseconds to about 50 microseconds. In one embodiment, each pulse may have pulse duration T_{w} of 20 microseconds. In various embodiments, the pulses 70 may be delivered at pulse frequency f₁ in the range of about 1 Hz to about 500 Hz. In certain embodiments, pulse frequency f₁ may be in the range of about 1 Hz to about 100 Hz. In one embodiment, pulse frequency f₁ may be for example 4 Hz.

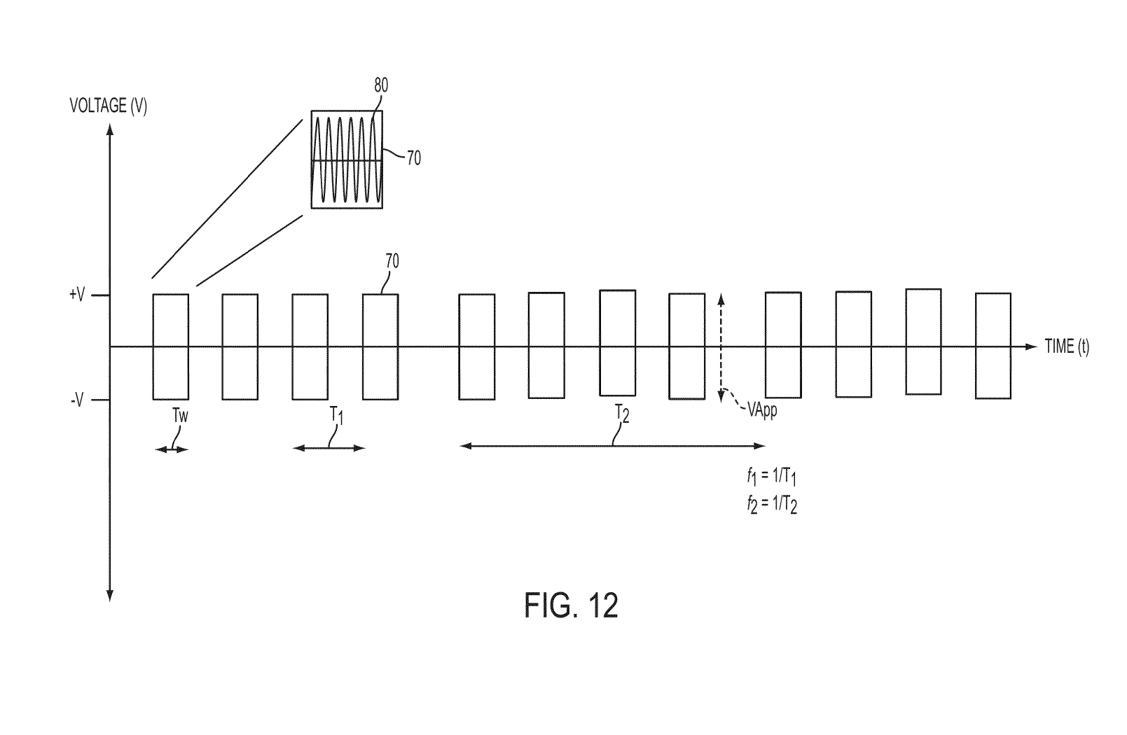

FIG. 12 is a graphical representation of multiple bursts of electrical pulses 70 generated and delivered by energy source 14 to promote wound healing at a wound site 12. Time (t) is shown along the horizontal axis and voltage (VAC) is shown along the vertical axis. In this embodiment, energy source 14 generates and delivers AC waveform 80 in three bursts. Each burst includes four pulses 70. Each pulse 70 has a duration T_{w} delivered at a pulse period T or a pulse frequency f₁=1/T₁. In addition, the bursts are spaced apart by sufficient time T_{b} to allow the temperature of the treated tissue to remain below a maximum temperature. The bursts repeat at a burst frequency f₂=1/T₂.

In various embodiments, the bursts may repeat at a burst frequency f₂ in the range of about 0.02 Hz to about 500 Hz. In certain embodiments, burst frequency f₂ may be in the range of about 1 Hz to about 100 Hz. The number of bursts generated and delivered in a treatment regimen may also be varied to maintain tissue temperature below a maximum temperature. The number of bursts may be in the range of about 1 to about 100 bursts. In certain embodiments, the number of bursts may be in the range of about 5 to about 50 bursts.

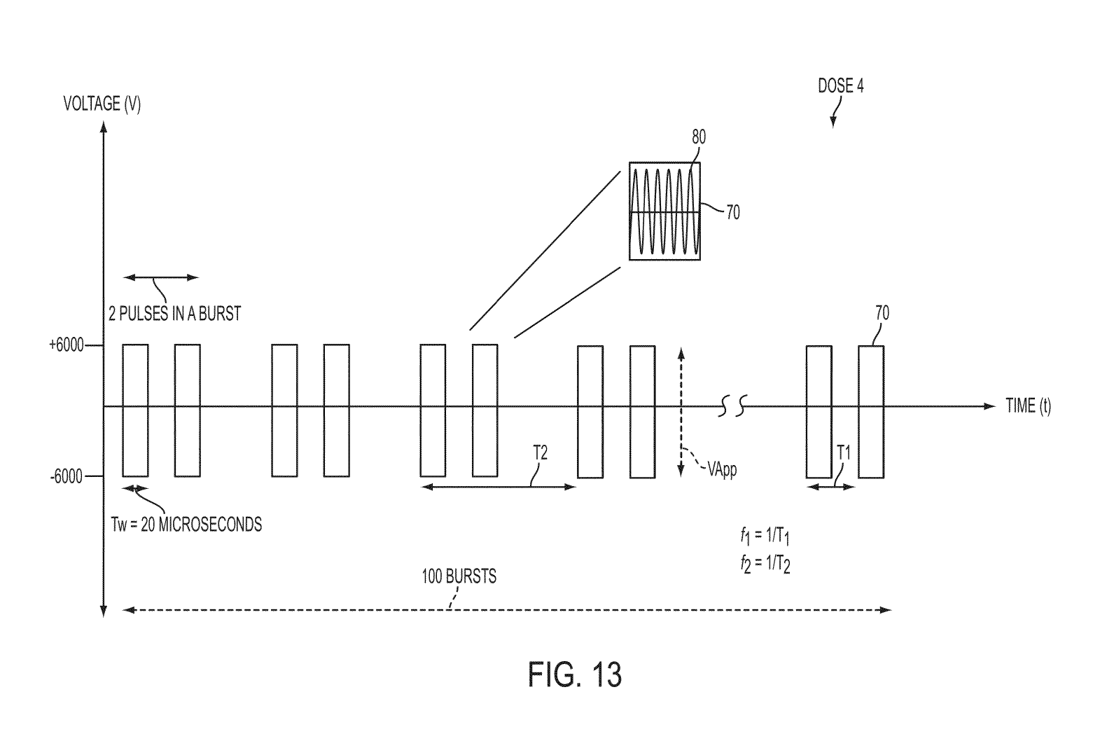

FIG. 13 is a graphical representation of a treatment regimen (Dose 4) used to promote wound healing at wound site 12. As illustrated in FIG. 13, AC waveform 80 has a fundamental frequency f of approximately 500 KHz and peak-to-peak voltage amplitude (VApp) of approximately 12,000 VAC. AC waveform 80 includes 100 bursts delivered at a burst period T₂ or repetition rate, frequency f₂=1/T₂, of approximately 0.5 Hz. Each burst includes 2 pulses. Each pulse 70 has a duration T_{w} of approximately 20 μs delivered at a pulse period T₁ or repetition rate, frequency f₁=1/T₁, of approximately 4 Hz.

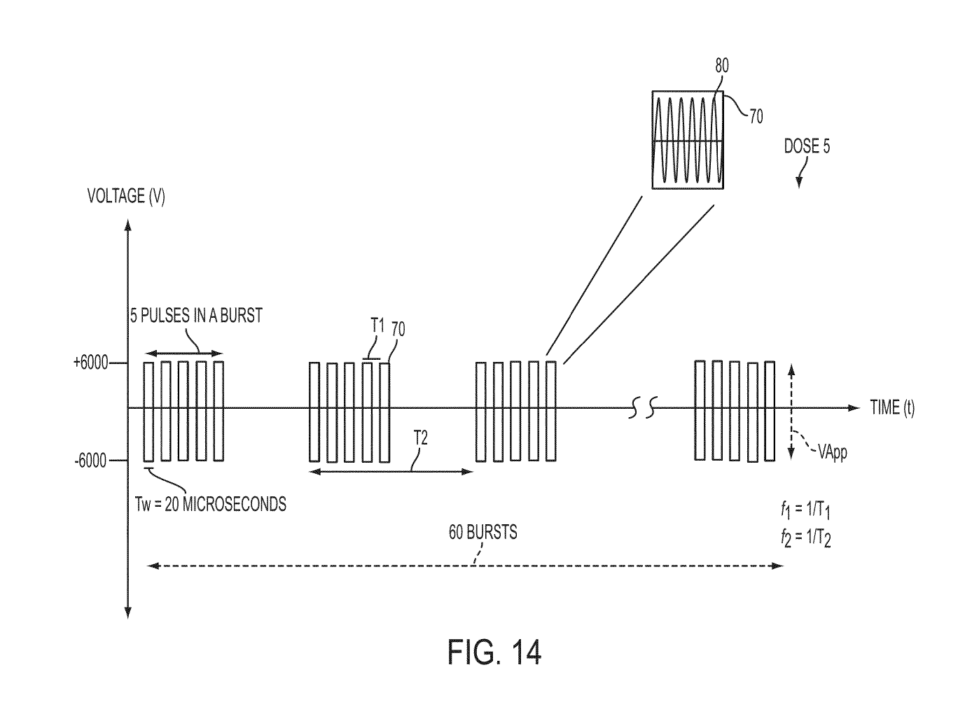

FIG. 14 is a graphical representation of a treatment regimen (Dose 5) used to promote wound healing at wound site 12. In this embodiment, energy source 14 generated and delivered AC waveform 80 having fundamental frequency f of approximately 500 KHz and peak-to-peak voltage amplitude (VApp) of approximately 12,000 VAC. The AC waveform 80 includes 60 bursts delivered at a burst period T₂ or repetition rate, frequency F₂=1/T₂, of approximately 0.2 Hz. Each burst includes five pulses 70. Each pulse has a duration T_{w} of approximately 20 μs delivered at a pulse period T₁ or repetition rate, frequency f₁=1/T₁, of approximately 4 Hz.

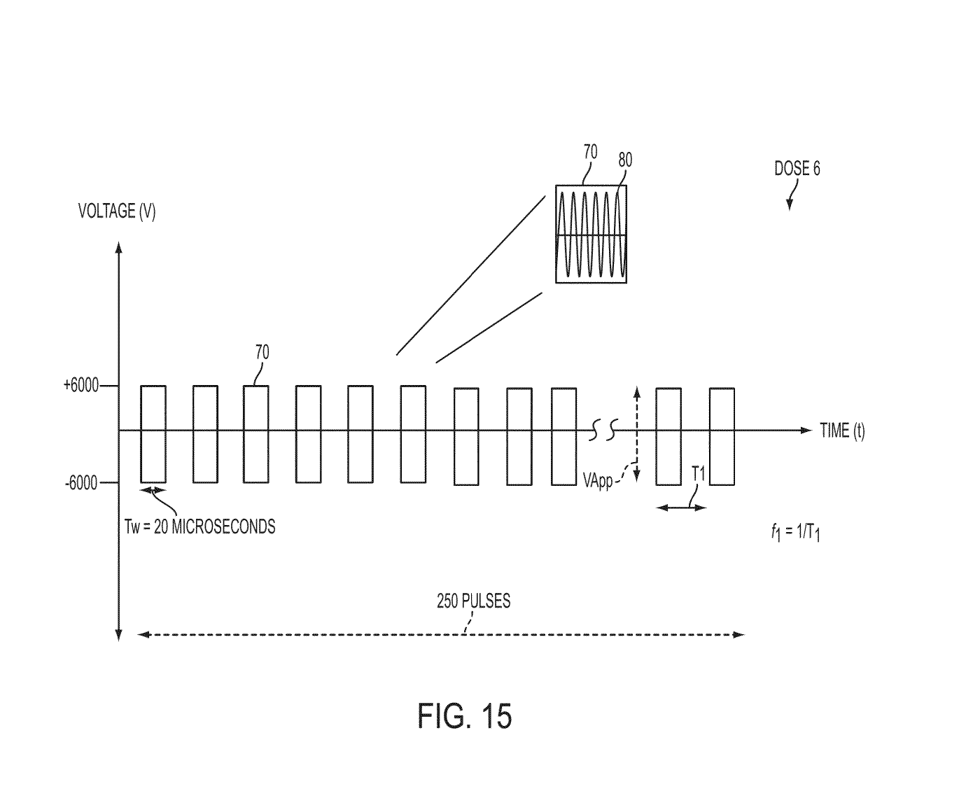

FIG. 15 is a graphical representation of a treatment regimen (Dose 6) used to promote wound healing at a wound site 12. In this embodiment, energy source 14 generated and delivered AC waveform 80 having fundamental frequency f of approximately 500 KHz and peak-to-peak voltage amplitude (VApp) of approximately 12,000 VAC. In this embodiment, AC waveform 80 includes 250 pulses. Each pulse has a duration T_{w} of 20 microseconds delivered at a pulse period T₁ or a pulse frequency, f₁=1/T₁, of 500 Hz.

Without wishing to be bound to any particular theory, energy source 14 may generate and deliver electric pulses 70 to promote wound healing at a wound site 12 with no or minimal heat applied to the treated tissue, and thus, may not destroy the cellular support structure or regional vasculature. In various embodiments, the temperature of the treated tissue may be maintained below or equal to 60° C. In other embodiments, the tissue temperature may be maintained below or equal to 50° C. In yet another embodiment, the tissue temperature may be maintained below or equal to 40° C.

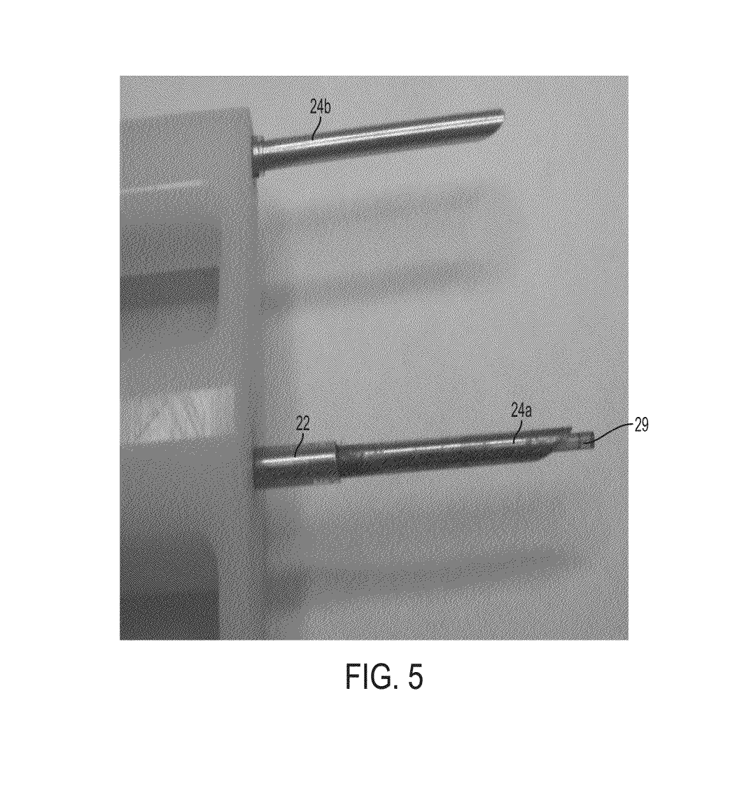

The temperature of treated tissue may be monitored using a temperature sensor as illustrated in FIG. 5. Transducers or sensors 29 may comprise a temperature sensor. In certain embodiments, the temperature sensor may be located in or proximate the electrosurgical device 20. The temperature sensor may be located within the handle 28. The temperature sensor may be located at the distal end of the flexible shaft 22. The temperature sensor may be located within the electrodes 24a,b. FIG. 5 is a photograph of an electrical ablation device comprising an optical temperature sensor 29 located in the electrode 24a at the distal end of the flexible shaft 22. In certain embodiments, the temperature sensor may be separate from the electrosurgical device 20.

According to certain embodiments, the temperature sensor may measure the temperature of the tissue at or around a wound site. The temperature sensor may measure the temperature of the tissue surrounding the electrodes. The temperature sensor may measure the temperature before, during, and/or after treatment. The temperature sensor may measure the temperature before a first sequence of electrical pulses is delivered to the tissue. The temperature sensor may measure the temperature after the first sequence of electrical pulses is delivered to the tissue. The temperature sensor may measure the temperature before a second sequence of electrical pulses is delivered to the tissue. The temperature sensor may measure the temperature after the second sequence of electrical pulses is delivered to the tissue.

The temperature sensor may provide feedback to the operator, surgeon, or clinician to apply an electric field pulse to the wound site. The feedback information provided by the transducers or sensors 29 may be processed and displayed by circuits located either internally or externally to the energy source 14.

In various embodiments, a wound site may be subjected to multiple doses of electrical pulses 70 in accordance with various embodiments described herein. In at least one embodiment, wound site 12 may be subjected to a first dose of electrical pulses 70 to promote wound healing during the Hemostasis stage. In addition, the wound site 12 may be subjected to a second dose of electrical pulses 70 to promote wound healing during the Inflammatory stage for example. In at least one embodiment, wound site 12 may be subjected to a first dose of electrical pulses 70 to sterilize the wound site 12. In addition, the wound site 12 may be subjected to a second dose of electrical pulses 70 to promote wound healing during the inflammatory stage.

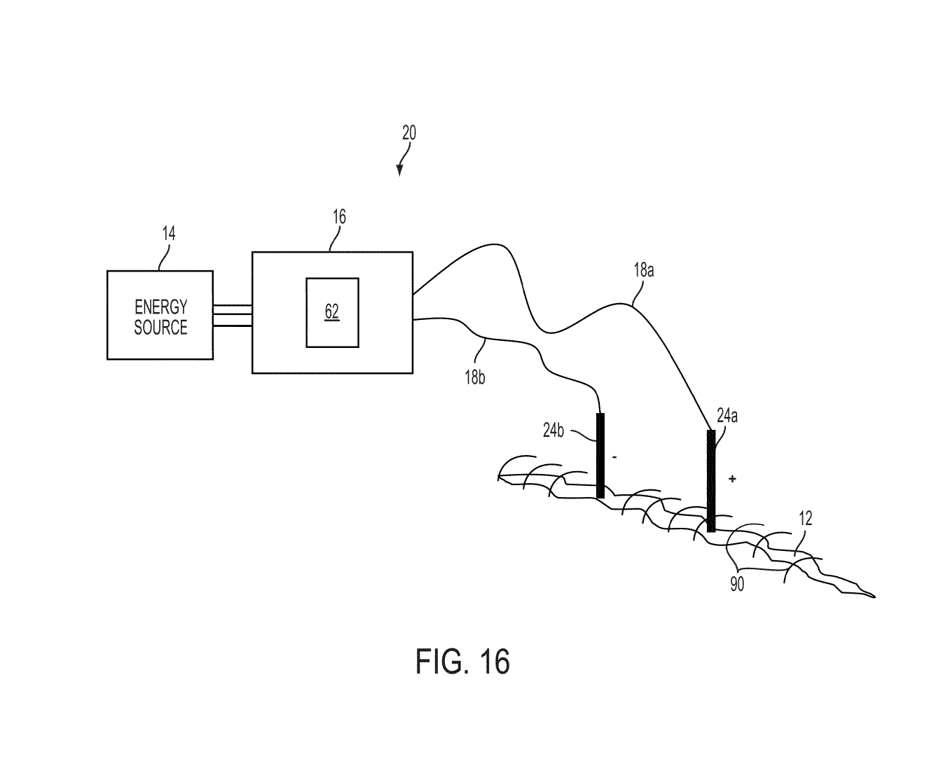

Referring to FIG. 16, a method for promoting wound healing is illustrated. In various embodiments, for example during a surgical procedure, wound site 12 may be treated with electrical pulses 70 to promote wound healing prior to and/or after closing the wound site 12 with a suture 90. As illustrated in FIG. 16, wound site 12 may be closed by suture 90. Electrodes 24a,b may be coupled to energy source 14, and positioned at wound site 12. Energy source 14 may then be configured to generate and deliver electrical pulses 70 in accordance with various embodiments described herein to promote wound healing at wound site 70.

In at least one embodiment, wound site 12 may be subjected to a first dose of electrical pulses 70, for example, to sterilize the wound site 12. The wound site 12 may then be closed by suture 90 as illustrated in FIG. 16. In addition, in certain embodiments, the closed wound site 12 may be subjected to a second dose of electrical pulses 70 to promote wound healing during the Inflammatory stage for example.

Those with ordinary skill in the art will appreciate that a variety of suturing devices and techniques may be utilized to close the wound site. Examples of commercially available sutures include PDS® sutures available from Ethicon, Inc., Somerville, N.J., Dexon® sutures available from United States Surgical Corporation, North Haven, Conn., Vicryl® (10/90) and Panacryl®. (95/5) sutures available from Ethicon, Inc., Somerville, N.J., Monocryl® sutures available from Ethicon, Inc., Somerville, N.J., and Maxon® sutures available from United States Surgical Corporation, North Haven, Conn.

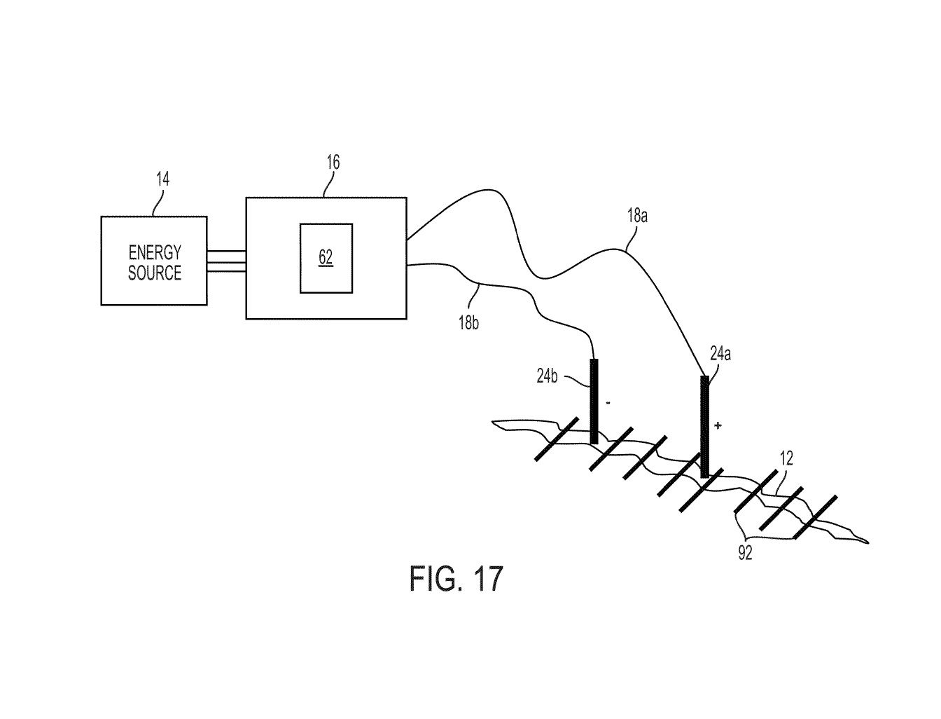

Referring to FIG. 17, a method for promoting wound healing is illustrated. In various embodiments, for example during a surgical procedure, wound site 12 may be treated with electrical pulses 70 to promote wound healing prior to and/or after closing the wound site 12 with a surgical stapler (not shown). As illustrated in FIG. 16, a surgical stapler may be operated to deploy staples 92 across the wound site 12 in order to close the wound site 12. Electrodes 24a,b may be coupled to energy source 14, and positioned at wound site 12. Energy source 14 may then be configured to generate and deliver electrical pulses 70 in accordance with various embodiments described herein to promote wound healing at wound site 70.

In at least one embodiment, wound site 12 may be subjected to a first dose of electrical pulses 70, for example, to sterilize the wound site 12. A surgical stapler may then be operated to deploy staples 92 across the wound site 12 in order to close the wound site 12. In addition, in certain embodiments, the closed wound site 12 may be subjected to a second dose of electrical pulses 70 to promote wound healing during the Inflammatory stage for example.

Those with ordinary skill in the art will appreciate that a variety of stapling devices and techniques may be utilized to close the wound site. Examples of commercially available staplers include PROXIMATE PX Fixed-Head Skin Stapler available from Ethicon, Inc., Somerville, N.J., and PROXIMATE PLUS MD Skin Stapler available from Ethicon, Inc., Somerville, N.J.

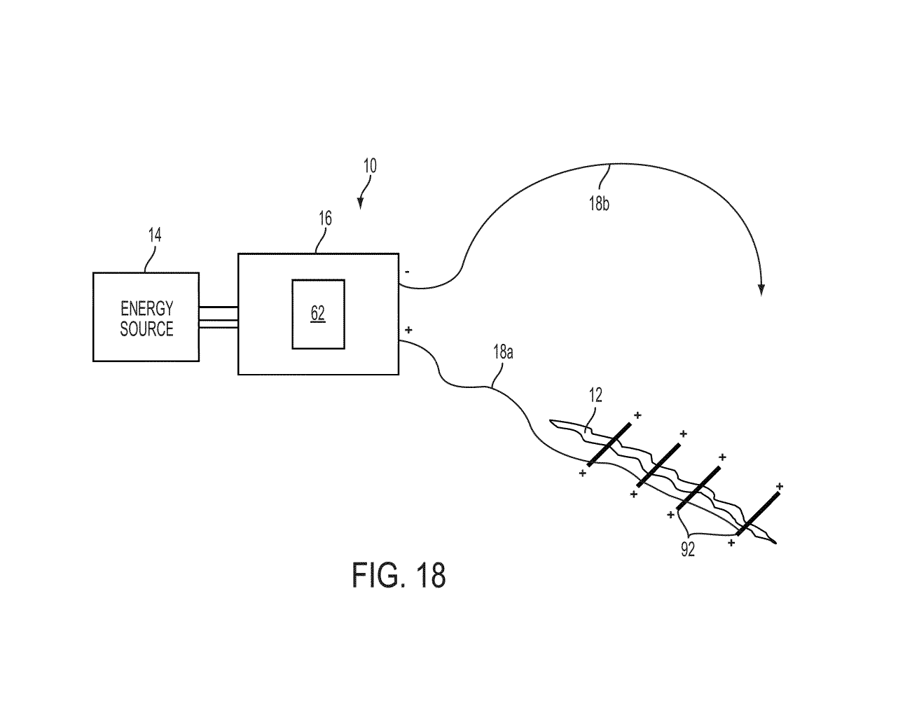

Referring to FIG. 18, a method and a system for treating wound site 12 is illustrated. In various embodiments, for example during a surgical procedure, a surgical stapler (not shown) may be operated to deploy staples 94 across wound site 12 in order to close the wound site 12. In at least one embodiment, staples 94 may be configured as a positive electrode, as illustrated in FIG. 18. Staples 94 may be electrically connected to electrical conductor 18a, which may be coupled to the positive terminal of energy source 14 through activation switch 62. A ground pad may be connected to electrical conductor 18b, which may be coupled to the negative terminal of the energy source 14 through the activation switch 62. Upon deploying staples 94, electrosurgical device 10 may be operated to deliver electrical pulses 70, in accordance with various embodiments described herein, to the wound site 12. Staples 94, in at least this embodiment, perform a dual function of closing the wound site 12 and acting as an electrode. In various embodiments, upon completion of the delivery of electrical pulses 70 to wound site 12, electrical conductor 18a may be severed or released from staples 94.

In some embodiments, staples 94 may be deployed individually. Alternatively, staples 94 may be housed in a staple cartridge (not shown) and deployed by a surgical stapler such as those described in U.S. Patent Publication No. US 2009/0209990 A1, filed Feb. 14, 2008, entitled “Motorized Surgical Cutting and Fastening Instrument Having Handle Based Power Source”, the entire disclosure of which is herein incorporated by reference. In at least one embodiment, the electrical conductor 18a can be disposed in a manner such that it is caught by the staples 94 as the staples 94 are released from the staple cartridge.

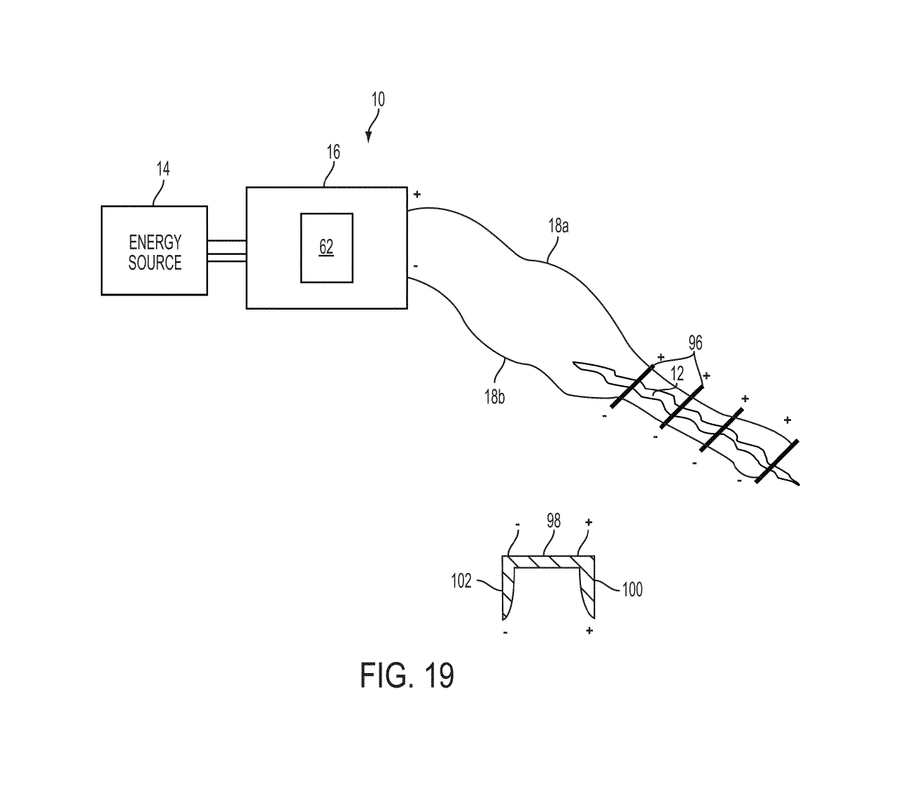

Referring to FIG. 19, a method and a system for treating wound site 12 is illustrated. In various embodiments, for example during a surgical procedure, a surgical stapler (not shown) may be operated to deploy staples 96 across wound site 12 in order to close the wound site 12. In at least one embodiment, at least one of staples 96 may include an insulated bridge portion 98, a first leg 100 acting as a positive electrode and a second leg 102 acting as a negative electrode. In other embodiments, all of staples 96, as illustrated in FIG. 19, may include insulated bridge portions 98, first legs 100 acting as positive electrodes, and second legs 102 acting as negative electrodes. The first legs 100 of staples 96 may be electrically connected to electrical conductor 18a, which may be coupled to the positive terminal of energy source 14 through activation switch 62. The second legs 102 of staples 96 may be electrically connected to electrical conductor 18b, which may be coupled to the negative terminal of energy source 14 through activation switch 62. Upon deploying staples 96, electrosurgical device 10 may be operated to deliver electrical pulses 70 to tissue at wound site 12, in accordance with various embodiments described herein, to the wound site 12. Staples 96, in at least one embodiment, perform a dual function of closing the wound site 12 and acting as electrodes. In various embodiments, upon completion of the delivery of electrical pulses 70, electrical conductors 18a and 18b may be severed or released from staples 96.

In some embodiments, staples 96 may be deployed individually. Alternatively, staples 96 may be housed in a staple cartridge similar to the staple cartridge described above in connection with staples 94. In at least one embodiment, the electrical conductor 18a and 18b can be disposed in a manner such that electrical conductor 18a and 18b are respectively caught by the first legs 100 and second legs 102 of staples 96 as the staples 96 are released from the staple cartridge.

The devices and systems disclosed herein or components thereof can be designed to be disposed of after a single use, or they can be designed to be used multiple times. In either case, however, the devices and systems may be reconditioned for reuse after at least one use. Reconditioning can include any combination of the steps of disassembly, followed by cleaning or replacement of particular elements, and subsequent reassembly. Upon cleaning and/or replacement of particular components, the device may be reassembled for subsequent use either at a reconditioning facility, or by a surgical team immediately prior to a surgical procedure. Those skilled in the art will appreciate that reconditioning may utilize a variety of techniques for disassembly, cleaning/replacement, and reassembly. Use of such techniques, and the resulting reconditioned device, are all within the scope of the present application.

It is preferred that at least some components of the devices and systems used herein are sterilized. This can be done by any number of ways known to those skilled in the art including beta or gamma radiation, ethylene oxide, steam, autoclaving, soaking in sterilization liquid, or other known processes.

Although various embodiments have been described herein, many modifications and variations to those embodiments may be implemented. For example, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1. A method for promoting wound healing in a patient, the method comprising:
positioning first and second electrodes at or near a wound site;
applying electrical pulses to tissue at the wound site, wherein the electrical pulses induce Irreversible Electroporation in cell membranes of the tissue at the wound site.

2. The method of claim 1, wherein the electrical pulses cause no or minimal thermal damage to extracellular matrix, and blood vessels at the wound site.

3. The method of claim 1, wherein the electrical pulses promote Hemostasis at the wound site.

4. The method of claim 1, further comprising maintaining the temperature of tissue at the wound site below a maximum temperature.

5. The method of claim 4, wherein the maximum temperature is equal to, or less than 60° C.

6. The method of claim 1, further comprising closing the wound site by suturing.

7. The method of claim 1, further comprising operating a surgical stapler to close the wound site by deploying staples across the wound site.

8. A method for promoting wound healing in a patient, the method comprising subjecting a wound site to electrical pulses that promote formation of a Hemostatic plug at the wound site.

9. The method of claim 8, wherein the electrical pulses promote formation of the hemostatic plug by increasing platelets count at the wound site.

10. The method of claim 8, wherein the electrical pulses cause no or minimal thermal damage to extracellular matrix, and blood vessels at the wound site.

11. The method of claim 8, further comprising maintaining the temperature of tissue at the wound site below a maximum temperature.

12. The method of claim 11, wherein the maximum temperature is equal to, or less than 60° C.

13. The method of claim 8, further comprising closing the wound site by suturing.

14. The method of claim 8, further comprising operating a surgical stapler to close the wound site by deploying staples across the wound site.

15. A method for promoting wound healing in a patient, the method comprising:
applying electrical pulses to a wound site, wherein the electrical pulses sterilize the wound site by inducing Irreversible Electroporation in foreign microorganisms at the wound site.

16. The method of claim 15, wherein the electrical pulses cause no or minimal thermal damage to extracellular matrix, and blood vessels at the wound site.

17. The method of claim 15, further comprises maintaining the temperature of tissue at the wound site below a maximum temperature.

18. The method of claim 17, wherein the maximum temperature is equal to, or less than 60° C.

19. The method of claim 15, further comprises closing the wound site by suturing.

20. The method of claim 15, further comprises operating a surgical stapler to close the wound site by deploying staples across the wound site.

21. A method for treating a wound site in a patient, the method comprising:
operating a surgical stapler to close the wound site by deploying staples across the wound site; and
applying electrical pulses to the wound site through the staples.

22. The method of claim 21, wherein the electrical pulses sterilize the wound site by inducing Irreversible Electroporation in foreign microorganisms at the wound site.

23. The method of claim 21, wherein the electrical pulses induce Irreversible Electroporation in cell membranes of tissue at the wound site.

24. The method of claim 21, wherein the electrical pulses promote formation of a Hemostatic plug at the wound site.

25. An electrosurgical system for treating a wound site in a patient, the electrosurgical system comprising:
an energy source; and
at least one staple deployable at the wound site, wherein the at least one staple is electrically coupled to the energy source, and wherein the at least one staple is configured to deliver energy from the energy source to tissue in electrical contact therewith.

26. The electrosurgical system of claim 25, wherein the at least one staple comprises a first electrically conductive portion, a second electrically conductive portion, and an electrically insulated portion between the first and second electrically conductive portions.

27. The electrosurgical system of claim 26, wherein the first electrically conductive portion comprises a positive electrode, and wherein the second electrically conductive portion comprises a negative electrode.

28. The electrosurgical system of claim 25, wherein the energy source is operative to generate and deliver pulses, and wherein the pulses induce Irreversible Electroporation in tissue in electrical contact with the at least one staple.
